(19) United States
(12) Patent Application Publication
(10) Pub. No.: US 2009/0076412 A1
(43) Pub. Date: Mar. 19, 2009

(54) Apparatus and Methods for Obtaining a Sample of Tissue

(75) Inventors: Robert Rioux, Ashland, MA (US); Jeffrey Cross, Charlestown, MA (US); Kathleen Kane, Brookline, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/209,515

(22) Filed: Sep. 12, 2008

Related U.S. Application Data
(60) Provisional application No. 60/972,010, filed on Sep. 13, 2007.

(51) Int. Cl. A61B 10/02

(52) U.S. Cl. 600/564

(57) ABSTRACT

Described herein are systems and methods for obtaining a sample of tissue with low power electrosurgical energy. The systems can include an apparatus for obtaining a sample of tissue from a patient having a cannula having a distal opening for receiving the sample of tissue. A wire, located proximate to the distal opening, can delivery energy to ablate tissue such that damage to the tissue sample is minimized.

### FIELD OF THE INVENTION

This application claims priority to Provisional Application Ser. No. 60/972,010 entitled “APPARATUS AND METHODS FOR OBTAINING A SAMPLE OF TISSUE” filed Sep. 13, 2007, which is incorporated herein by reference.

The present invention relates generally to the field of surgery. More particularly, the present invention relates to a surgical apparatus and a method for obtaining a sample of tissue from a patient.

### DESCRIPTION OF THE BACKGROUND ART

It is often necessary for a physician, as part of establishing or confirming a diagnosis, to obtain a sample of tissue from a patient for analysis. As a result, a variety of biopsy tools have been developed to facilitate tissue sampling. Such biopsy tools generally include a mechanical cutting device and a tissue sample receiving chamber. For example, core needle biopsy devices generally include a hollow needle coupled with a spring-actuated-type cutting mechanism. The needle is advanced into a patient and the cutting mechanism is fired to resect a tissue sample.

One drawback of such devices is the danger of accidentally injury while handling or operating such devices. The sharp cutting surfaces and spring activated mechanisms can result in needle stick injuries, a common problem associated with the handling or operating medical devices with sharp surfaces. Unfortunately, these injuries may expose the victim to infectious agents, including the hepatitis B, hepatitis C, and human immunodeficiency viruses.

Therefore, a need remains for improved biopsy devices, particularly biopsy apparatus and methods that safeguard physicians and/or other medical staff.

### SUMMARY

Described herein are methods and devices for obtaining a sample of tissue using low power electrosurgical energy. In one embodiment, a biopsy device is provided that includes an electrically conductive energy delivery member positioned on a distal portion thereof. The biopsy device and energy delivery member do not include mechanical cutting surfaces, but rather the biopsy device can harvest a tissue sample with low power electrosurgical energy. Conventionally, electrosurgical energy has been used to coagulate or cauterize tissue. The energy, while effective at reducing blood loss, resulted in tissue damage that would make tissue samples inadequate for testing. The biopsy devices provided herein uses low power electrosurgical energy to cut away a tissue sample. The low power electrosurgical energy minimizes damage to tissue samples, while eliminating the need for mechanical cutting surfaces and the associated risk of accidental injury.

In one embodiment, a biopsy device includes a cannula having an elongated body extending from a proximal end to a distal end and an inner lumen extending from a distal opening for receiving the sample of tissue. The elongate body can be defined by a sidewall. The distal end of the device includes a first wire comprising an electrically conductive material, which is in electrical communication a power source.

In one aspect, the wire has a sufficiently small size such that tissue can be incised when low power electrical energy is transmitted to the wire. In one embodiment, the first wire is sized to allow tissue penetration when the electrosurgical energy delivered to the first wire does not exceed about 10 joules per second. In another embodiment, the first wire is sized to allow tissue penetration when the electrosurgical energy delivered to the first wire is in the range of about 1 to 7 joules per second.

In another aspect, the wire has a size smaller than the distal end of the elongate body. For example, the elongated body can have a first wall thickness at the distal opening and the first wire can have a cross-sectional dimension, transverse to the elongate body, which is less than the first wall thickness. In addition, or alternatively, the wire can cover only a portion of the surface area of the distal end of the elongate body.

The cannula can have a variety shapes and sizes depending on the intended use of the biopsy device. For example, the cannula can have a generally cylindrical body and a non-tissue penetrating shaped distal end.

In another embodiment, the cannula can be adapted to cooperate with an introducer. For example, the biopsy device can further comprise an introducer configured to be inserted into an inner lumen of the cannula. The introducer can include a body having a size and shape corresponding to the inner lumen of the cannula, such that the introducer can slide proximally and distally within the inner lumen. In one aspect, the introducer can include an elongated body and a blunt distal tip.

In another aspect, the introducer is configured to electrosurgically cut tissue. For example, the introducer can include a second wire affixed to the distal tip thereof. In use, low power electrosurgical energy can be delivered to the second wire.

In yet another aspect, the cannula can cooperate with a tissue cutter. For example, the sidewall of the cannula can include an aperture proximate to the distal end of the cannula. The aperture can receive a flexible tissue cutter and can direct the tissue cutter across the inner lumen of the cannula. In one aspect, the tissue cutter is also configured to cut tissue with low power electrosurgical energy. For example, a third wire can be positioned on the distal end of the tissue cutter.

In another embodiment, a system for obtaining a sample of tissue from a patient is provided. The system includes a cannula, including an elongated body extending from a proximal end to a distal end and an inner lumen extending from a distal opening for receiving the sample of tissue. A first wire is mated to the distal end of the elongated body. The system further comprises an introducer positioned within the inner lumen of the cannula and including an elongated body and a blunt distal tip. A second wire is mate to the distal tip of the introducer. The system can also include a tissue cutter configured to incise a tissue sample collected within the inner lumen of the cannula. The tissue cutter can include a third wire adapted to cut tissue with low power electrosurgical energy.

Further provided herein are methods for obtaining a sample of tissue using low power electrosurgical energy. In one embodiment, a method for obtaining a sample of tissue includes the steps of providing a biopsy device comprising a cannula including an inner lumen extending from a distal opening for receiving the sample of tissue and a wire adapted to deliver low power electrosurgical energy. An energy source can provide electrosurgical energy to the wire and the biopsy device can be moved through a tissue mass to collect a tissue sample within the inner lumen.

In another embodiment, the cannula cooperates with an introducer configured for receipt within the inner lumen of the cannula. The distal end of the introducer can include a second wire for delivering electrosurgical energy.

In one aspect, the introducer is adapted to move from a tissue penetrating configuration, where the distal end of the introducer and the second wire extend from the distal end of the cannula, to a tissue sampling configuration where the introducer is recessed within the inner lumen. The method can include the step of positioning the biopsy device in a tissue penetrating configuration and supplying electrosurgical energy to the second wire. The biopsy device can then electrosurgically incise tissue while moving to a target tissue location.

The method can further include moving the introducer into a tissue sampling configuration and supplying electrosurgical energy to the first wire. The cannula can then collect a tissue core within the inner lumen.

In yet another aspect, the cannula can cooperate with a tissue cutter to cut the tissue core away from a tissue mass. For example, the method can further comprise the step of moving a tissue cutter across the width of the cannula to cut the tissue core within the cannula.

It is to be understood that both the foregoing general description and the following detailed description are exemplary and explanatory only and are not restrictive of the invention in any way.

### BRIEF DESCRIPTION OF THE DRAWINGS

The accompanying drawings, which are incorporated in and constitute a part of this specification, illustrate exemplary embodiments of the invention and together with the description, serve to explain the principles of the invention. It is to be understood that the drawings are exemplary and explanatory only and are not restrictive of the invention in any way.

FIG. 1 is a perspective view of an exemplary system for obtaining a sample of tissue according to an embodiment of the invention;

FIG. 2 is a partial perspective view of one embodiment of a biopsy device disclosed herein;

FIG. 3A is a distal view of the biopsy device of FIG. 2;

FIG. 3B illustrates another embodiment of the biopsy device of FIG. 3A;

FIG. 4A is a cross-sectional schematic view of the biopsy device of FIG. 3A;

FIG. 4B illustrates another embodiment of the biopsy device of FIG. 4A;

FIG. 4C illustrates another embodiment of the biopsy device of FIG. 4A;

FIG. 4D illustrates another embodiment of the biopsy device of FIG. 4A;

FIG. 4E illustrates another embodiment of the biopsy device of FIG. 4A;

FIG. 5A is a perspective view of an introducer disclosed herein;

FIG. 5B is a distal view of the introducer of FIG. 5A;

FIG. 6A is a perspective view of a tissue cutter disclosed herein;

FIG. 6B is a perspective view of another embodiment of the tissue cutter of FIG. 6A;

FIG. 7 is a cross-sectional view of a tissue cutter extending through an aperture in a biopsy device disclosed herein;

FIG. 8A is a cross-sectional view a biopsy system comprising a cannula, an introducer, and a tissue cutter;

FIG. 8B is a cross-sectional view of the system of FIG. 8A with the introducer in a tissue sampling configuration;

FIG. 8C is a cross-sectional view of the system of FIG. 8A with the tissue cutter advanced through an aperture in the cannula; and

FIG. 9 is a perspective view of one embodiment of a handle disclosed herein.

### DETAILED DESCRIPTION

Described herein are systems and methods for sampling tissue, including tissue sampling devices capable of harvesting a tissue sample using low power electrosurgical energy. The devices can include an electrically conductive wire positioned on a tissue contacting surface of a cannula body. In one aspect, the wire is sized to provide low power electrosurgical cutting energy such that the cannula can harvest tissue while minimizing tissue sample damage. In another aspect, an electrosurgical introducer and/or tissue cutter can be used with the cannula. The electrosurgical devices can provide an effective biopsy system that reduces the chance of medical personal accidentally cutting themselves with a tissue penetrating surface.

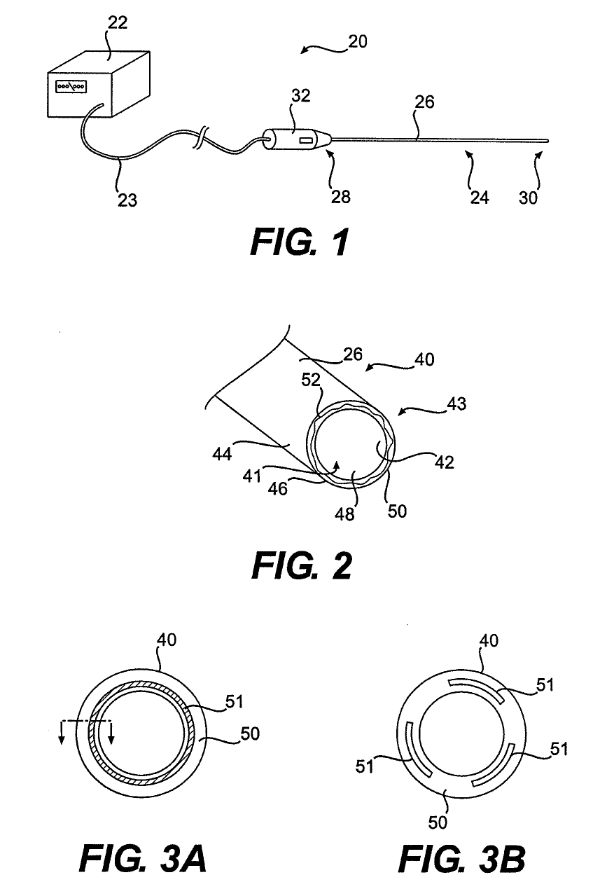

FIG. 1 illustrates one embodiment of a system 20 for obtaining a sample of tissue using low power electrosurgical energy. The exemplary embodiment includes an energy source 22 in electrical communication with a biopsy device 24. In one aspect, device 24 includes an elongate body 26 extending between a proximal end 28 and a distal end 30. Proximal end 28 can be mated with a handle 32 adapted to facilitate control of the various element of system 20, while distal end 30 is adapted to collect a tissue sample from a target tissue area.

Energy source 22, in one embodiment, provides electrosurgical energy in a controlled manner via a cable 23 to handle 32 and/or biopsy device 24. Energy source 22 can include the variety of conventional electrosurgical energy generators and/or controllers. In one aspect, energy source 22 is capable of delivering electrosurgical energy, such as radio frequency (“RF”) energy over a range of power levels. The energy source and/or other portions of system 20 can include a power control mechanism that limits and/or controls the amount of energy delivered to biopsy device 24. Energy source 22 and handle 32 are discussed in more detail below.

Biopsy device 24, in one aspect, includes an elongate body 26 that allows a surgeon to sample tissue at a distance from a tissue target site. The size and shape of elongate body 26 can be varied depending on the type and location of tissue to be sampled. While body 26 is illustrated as straight, the elongate body could alternatively have a curved or steerable body to facilitate placement of distal end 28 within a target tissue area.

Elongate body 26 can be formed by a cannula having a distal end adapted to receive a tissue sample. FIG. 2 provides a distal, perspective view of one embodiment of a cannula 40 including an open distal end 48 and an inner lumen 41 defined by a sidewall 46. The sidewall includes an inner surface 42 and an outer surface 44.

As shown in FIG. 2, cannula 40 can have a generally cylindrical shape. However, cannula 40 can include a variety of alternative cross-sectional shapes, including a circular, triangular, oval, rectangular, or irregular cross-sectional shape. In addition, the inner and/or outer surface 42, 44 can have varying cross-sectional shapes along the length of the cannula. Opening 48 can have the same or a different shape and size as cannula 40. However, in one embodiment, the distal portion of inner lumen 41, including opening 48, has a uniform cross-sectional shape along at least a portion of the length of the cannula. One skilled in the art will appreciate that cannula 40 can have a variety of alternative shapes and sizes depending on the intended use of system 20.

Regardless of the shape of cannula 40, the cannula preferably does not include any exposed tissue cutting surfaces. Conventional biopsy devices generally include a mechanical cutting surface that creates a tissue incision when force is applied to the cutting surface. The cannula described herein does not include any such surface. For example, instead of a sharp distal surface 50, the distal surface 50 of sidewall 46 can be blunt.

To create a tissue incision, in one aspect, the distal surface 50 of sidewall 46 includes an electrosurgical energy delivery member 52. The energy delivery member can be positioned to contact tissue and electrosurgically resect tissue with low power energy. In use, a surgeon can activate biopsy device 24 and deliver energy to delivery member 52. Cannula 40 can then be moved through a tissue mass to collect tissue within inner lumen 41.

Formerly, it was believed that electrosurgical energy could not be used to resect tissue as part of a tissue sampling procedure, because the electrosurgical energy would severely damage the collected tissue. The only use for electrosurgical energy with biopsy devices was to cauterize tissue. Conversely, the biopsy devices provided herein uses an energy delivery member that allows a user to harvest a useful tissue sample with low power electrosurgical energy.

The small size of energy delivery member 52 allows biopsy device 24 to incise tissue at low power while minimizing damage to the tissue sample. In one aspect, the volume of energy delivery member 52 allows tissue penetration when the electrosurgical energy delivered by the energy source to the first energy delivery member does not exceed about 15 joules per second. In another aspect, the energy delivery member 52 is sized to allow tissue penetration when the electrosurgical energy delivered to the energy delivery member is in the range of about 1 to 10 joules per second, or, more preferably, in the range of about 1 to 7 joules per second.

The volume of the energy delivery member which will allow ablation at low power depends on a variety of factors, such as, the size of the cannula and the type of tissue. Thus, the volume of the energy delivery member at the distal end of the cannula can vary.

As shown in FIG. 2, the small size of energy delivery member 52 results in the energy delivery member covering only a portion of distal surface 50. In one aspect, the energy delivery member occupies less than the full width of sidewall 46 or less than the full area of distal surface 50. For example, depending on the configuration of the cannula, the energy delivery member 52 can occupy less than about 80% of the distal surface area of cannula 40, often less than about 60%, or even less than about 40% of the distal surface area of cannula 40. Similarly, the energy delivery member can have a width that does not exceed about 75% of the width of the sidewall 46, or even 50% of the width of sidewall 46.

In one embodiment, energy delivery member 52 is an electrically conductive wire. FIG. 3A illustrates a front view of a cannula 40 with wire 51 mated to the distal end of the elongated body of cannula 40. When the electrosurgical energy is applied to the wire, the biopsy device can cut through tissue adjacent to distal surface 50.

In one aspect, wire 51 extends around the full circumference of open distal end 48. Alternatively, wire 51 can extend around a part of the circumference of distal opening 48. For example, FIG. 3B illustrates the exposed portion of wire 51 in a non-contiguous configuration. In another aspect, instead of a single wire, separate wires can extending around distal opening 48. For example, one, two, or more than two separate wires can extend from a common electrosurgical energy source and ablate tissue adjacent to the distal end of the cannula 40.

Wire 51 can extend from distal surface 50. For example, the wire can extend about 0.1 mm to 2 mm from the distal end of the biopsy device, and preferably 0.2 to 1 mm from the distal end of the biopsy device. In another embodiment, distal end of wire 51 is generally coplanar with the distal surface of the biopsy device.

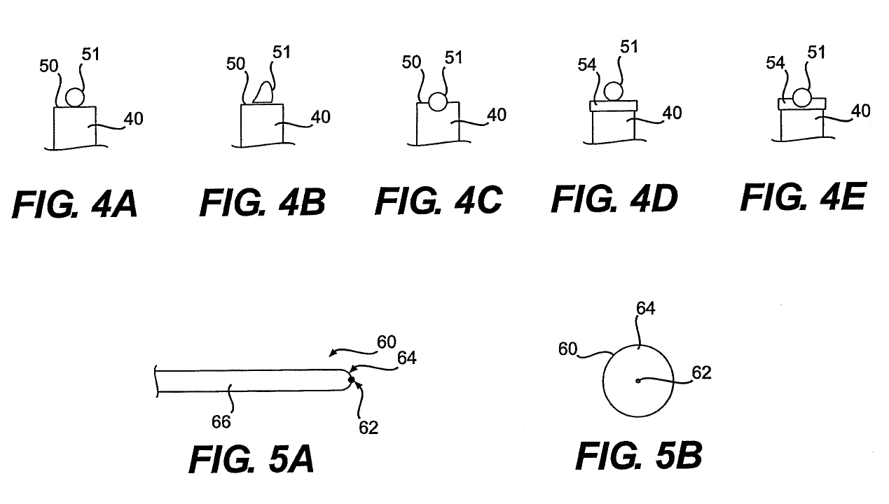

In one embodiment, wire 51 does not have any surfaces or edges sharp enough to accidentally penetrate tissue mechanically while a user is handling or operating the biopsy device. For example, wire 51 can have a circular, elliptical, polygonal, irregular, and/or triangular cross-sectional shape defining a blunt distal surface. FIGS. 4A-4E illustrate cross-sectional views of various exemplary configurations of the distal end of the cannula 40 and wire 51. Where wire 51 has a generally circular cross-sectional shape, the wire size can be equal to or less than about 16 gauge. In another aspect, the wire can be equal to or less than about 18 gauge.

Wire 51 can be mated with cannula 40 in a variety of ways including, for example, welding, adhering, and/or mechanically engaging. As illustrated in FIGS. 4A and 4B, the wire can be adhered to the surface of the distal end of the cannula 40. Alternatively, as illustrated in FIG. 4C, the wire can be partly recessed in the distal end of the cannula 51. For example, wire 51 can be molded into the distal end of the cannula.

Cannula 40 can be formed, at least in part, of an electrically insulative material such that the cannula generally does not participate in tissue cutting. For example, the cannula can be formed from fluorinated ethylene propylene (FEP), Polyetheretherketones (PEEK), and/or polytetrafluoroethylene (PTFE). Alternatively, or additionally, an insulative member can be positioned between wire 51 and cannula 40. The insulative member can assist with mating wire 51 (e.g., insulating member can be an adhesive) and/or wire 51 can be mated to insulative member 54. As illustrated in FIGS. 4D and 4E, the wire 51 may be affixed to, or partly molded into, an insulative member.

Wire 51 can include an exposed portion for delivering energy to tissue and an unexposed or insulated portion that is in contact with cannula 40 or insulative member 54. Depending on the shape of wire 51 and the method of affixing wire 51 to cannula, the amount of exposed surface can vary. In one embodiment, the exposed surface of the wire relative to the unexposed surface of the wire in contact with the distal end of the cannula or insulative member is in the range of about 80% and 20%. In another embodiment, the exposed surface of the wire at the distal surface of cannula 40 relative to the unexposed surface of the wire is at least about 70%, at least about 60%, and preferably at least about 50%. Having a higher percentage of wire surface exposed can minimized the total volume of wire positioned around the distal end of the cannula and reduce the power needed to resect tissue.

As discussed herein, the volume of energy delivery member 52 refers to the volume, exposed and unexposed, of the energy delivery member positioned at the distal end of cannula 40. The energy delivery member can be connected to energy source 22 and/or handle 32 via a transmission cable (not illustrated) that extends in, along, and/or through the cannula. The volume of the electrically conductive portion of the transmission cable can also be minimized. In one aspect, the transmission cable is defined by a wire with a diameter less than about 12 gauge. In another aspect, the wire has a size equal or less than about 16 gauge.

In another embodiment of the biopsy device described herein, cannula 40 can cooperate with an introducer during insertion of the biopsy device into a tissue mass. In one aspect, the introducer is adapted to ablate tissue rather than mechanically cut tissue. FIG. 5A illustrates an exemplary introducer 60 with a wire 62 is positioned on the distal portion thereof. Wire 62 can include the various features of wire 51 discussed above. For example, wire 62 can have a small volume which allow wire 62 to cut tissue with low power electrosurgical energy.

The introducer can be configured to allow the biopsy device to cut through tissue in a non-sampling configuration and then change to a sampling configuration. For example, in the non-sampling configuration the introducer can be positioned within the inner lumen of the cannula such that the introducer blocks the open distal end of the cannula. In addition, the distal portion of the introducer, including wire 62, can extend from the distal opening in the cannula.

With the introducer positioned within the distal cannula opening, the introducer and cannula can move through tissue together. Once the biopsy device reaches the target tissue region, the introducer can be retracted (i.e., moved proximally) relative to the cannula to open the inner lumen for receipt of a tissue sample. The cannula can then be advanced to collect the tissue sample.

The introducer can have a variety of shapes and sizes, however in one embodiment, the outer surface of the introducer generally conforms to at least a portion of the inner surface of the cannula. In one aspect, the distal end of introducer does not have a mechanical tissue cutting surface and instead has a generally blunt distal end. As shown in FIG. 5A, introducer 60 can include a semispherical distal surface 64 positioned on an elongate body 66. One skilled in the art will appreciate that the introducer can have a variety of non-tissue cutting configurations including a variety of differently shaped distal ends.

In one aspect, introducer can slide within cannula to move between the tissue penetrating configuration and the tissue sampling configuration. The size and shape the inner lumen can control movement of introducer 60 such that the introducer moves proximally and distally with respect to the cannula. For example, the inner lumen of the cannula and the outer surface of introducer body 66 can mate with a clearance fit. By varying the relative dimensions of the inner lumen and the introducer, the force required to move the introducer can be controlled. In addition, materials used to form the inner surface 42 of lumen 41 and/or the surface of the body 66 of the introducer can be varied to control the amount of friction between the introducer and the cannula. In one aspect, a low friction material can facilitate movement of the introducer through inner lumen 41.

In one embodiment, a user can move the device between the tissue penetrating and tissue sampling configuration using handle 32. For example, the introducer and cannula can mated with handle 32 such that handle 32 can be actuated to move cannula and introducer relative to one another. In addition, handle 32 can be configured to allow a user to lock the introducer relative to the cannula during insertion of the introducer and cannula through tissue.

The introducer can be formed from a variety of materials including medical grade metals and polymers. Body 66 can be formed of generally rigid materials to minimize deflection of the introducer as the biopsy device is moved through tissue. Alternatively, body 66 can comprise semi-rigid or flexible materials where the inner lumen of cannula 40 is sized to support body 66. In one aspect, body 66 does not participate in tissue ablation and is formed of electrically insulative materials. In addition, or alternatively, an insulating member can be positioned between wire 62 and body 66.

In the tissue penetrating configuration, the distal surface 64 of introducer 60 can extend from the distal end of cannula 40 to expose wire 62. For example, wire 62 can be positioned on the distal most surface introducer 60 such that wire 62 is exposed when a distal portion of the introducer is advanced through opening 48 of the cannula.

The wire extending from introducer 60 can have a variety of configurations. In one aspect, shown in FIGS. 5A and 5B, only the tip of wire 62 is exposed. The distal end of the wire can be coplanar with the distal end of the introducer. Alternatively, a portion of the wire can be exposed at the distal end of the introducer. For example, the wire can extend along a portion of the distal surface of introducer 60, or alternatively, wire 62 can extend from distal surface 64.

In one aspect, wire 62 can draw electrical current directly from source 22 via a transmission cable as discussed above with respect to wire 51. Wire 62 can be in electrical contact with the same or a different transmission cable as wire 51. In one aspect, a transmission cable can extend through, or run along, introducer 60. In another aspect, at least a portion of body 66 of introducer 60 can be formed of electrically conductive materials such that electrosurgical energy is transmitted through introducer 60.

In yet another embodiment of the biopsy device described herein, cannula 40 can cooperate with a tissue cutter. When the biopsy device is in a tissue sampling configuration, cannula is advanced into a target tissue mass. A tissue core is received in the inner lumen, which is still attached to the tissue mass. The tissue cutter works with the cannula to sever the tissue core from a tissue mass. One skilled in the art will appreciate that a variety of tissue cutters can cooperate with the cannula depending on the configuration of the distal portion of the biopsy device.

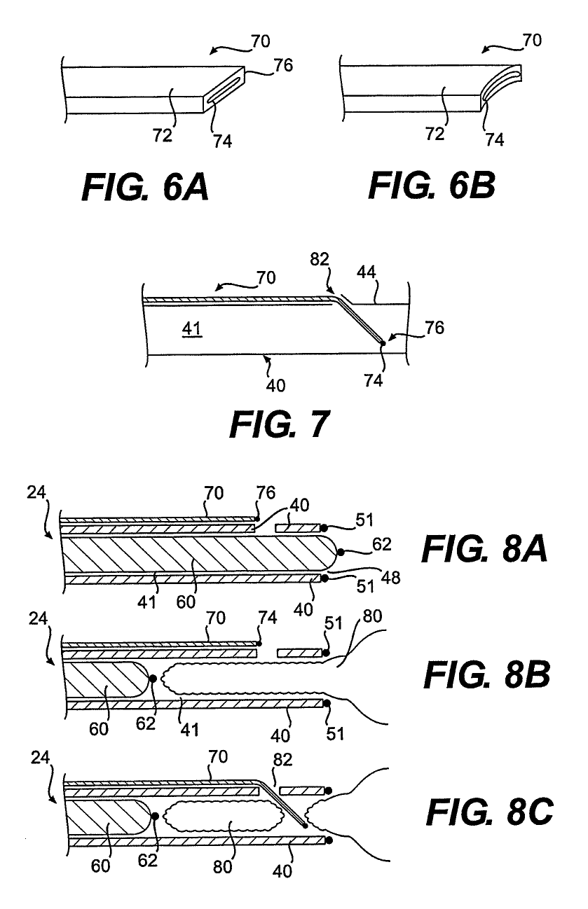

In one aspect, the tissue cutter is a flexible shaft with a non-sharp distal end. In use, the tissue cutter can extend through an aperture in the sidewall of the cannula and sever the tissue core. FIGS. 6A and 6B illustrate a tissue cutter 70 having a flexible body member 72 with a non-sharp distal end 76. Distal end 76 includes a wire 74 adapted to receive electrosurgical energy and cut a tissue sample positioned in, or adjacent to, inner lumen 41 of cannula 40. Wire 74 can include the various features of wires 51, 62 described above.

In one embodiment, flexible body member 72 is positioned along the outer surface 44 of cannula 40 with distal end 76 positioned for receipt in the aperture of sidewall 46 of cannula 40. Moving the flexible body member distally relative to the cannula moves distal end 76 of the tissue cutter through the aperture. The aperture can have a shape and size configured to facilitate directing the flexible body member at an angle with respect to the elongate body of cannula 40.

For example, as shown in FIG. 7, aperture 82 causes tissue cutter 70 to bend and directs distal end 76 and wire 74 across the width of lumen 41. As the tissue cutter is advanced, wire 74 cuts tissue by delivering electrosurgical energy. The result is a tissue sample positioned within inner lumen 41.

The size and shape of tissue cutter 70 can vary depending on the configuration of cannula 40. In one aspect, (illustrated in FIG. 6B) flexible body member 72 can have a shape that generally corresponds to the outer surface 44 of cannula 40. In order to minimize tissue damage as biopsy device 24 is moved through a tissue mass, flexible body member 72 can have a low profile. For example, the thickness of flexible body member 72 extending from the outer surface of cannula 40 is minimized. In addition, flexible body member 72 can be positioned within a recess (not illustrated) in outer surface 44 or within sidewall 46 of cannula 40.

In use, the proximal end of tissue cutter can be mated with handle 32, as described in more detail below, to allow a surgeon to manipulate the tissue cutter relative to the cannula. Alternatively, the proximal end of the tissue cutter can include features to allow a user to directly control the tissue cutter.

FIGS. 8A-8C illustrate the distal end of biopsy device 24 with introducer 60 and a tissue cutter 70. FIG. 8A illustrates introducer 60 in a non-sampling/tissue penetrating configuration suitable for insertion through a tissue mass. Introducer 60 is positioned within inner lumen 41 of cannula 40 and extends out of the distal opening 48. Generally, introducer 60 can be sized and shaped to extend from cannula such that wire 62 on introducer 60 can deliver electrosurgical energy and allow device 24 to penetrate tissue.

FIG. 8B illustrates the biopsy device 24 is a tissue sampling configuration. Introducer 60 has been partially retracted from the cannula 40 and the cannula has been advanced into a tissue mass to cut a sample of tissue 80. As shown, a tissue sample 80 is positioned within inner lumen 41, but is not fully resected. FIG. 8C illustrates cutting member extending through aperture 82 and completing the excision of the sample of tissue 80.

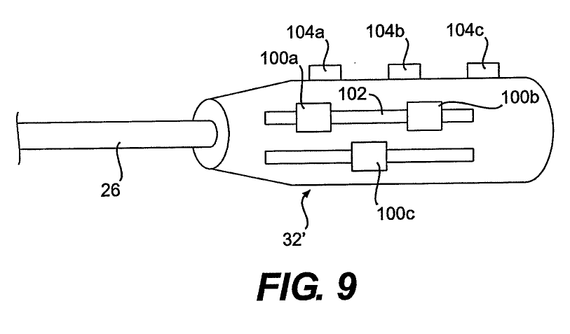

Handle 32, as mentioned above, can be configured to mate with cannula 40, introducer 60, and/or cutter 70. FIG. 9 illustrates handle 32′ mated with elongate body 26 which can comprise cannula 40, introducer 60, and/or tissue cutter 70. In one aspect, handle 32′ includes mechanical actuators 100a, 100b, 100c for controlling cannula 40, introducer 60, and/or cutter 70. The handle can be formed from a durable and rigid material, such as medical grade plastic, and is ergonomically molded to facilitate manipulation of cannula 40, introducer 60, and/or cutter 70.

In one aspect, cannula 40 and introducer 60 are slidably mounted within handle 32′ such that they can coaxially and independently move relative to one another. Mechanical actuators 100a and 100b can be coupled to cannula and introducer such that moving the mechanical actuators along pathway 102 moves the distal end of cannula 40 and introducer 60. Similarly, mechanical actuator 100c can be mated with cutter 70 so that the cutter can be independently actuated. One skilled in the art will appreciate the mechanical actuators 100a, 100b, 100c are merely illustrative of one of the various ways in which cannula, introducer, and cutting member can be manipulated.

In one embodiment, handle 32′ is electrically mated with an energy source. Buttons 104a, 104b, 104c can control delivery of electrosurgical energy to wires 51, 62, and/or 74.

The energy source can be a conventional RF power supply that operates at a frequency in the range from 300 KHz to 9.5 MHz, with a conventional sinusoidal or non-sinusoidal wave form. Such power supplies are available from many commercial suppliers, such as Valleylab, Aspen, and Bovie. Most general purpose electrosurgical power supplies, however, operate at higher voltages and powers than would normally be necessary or suitable for tissue ablation. Thus, such power supplies would usually be operated at the lower ends of their voltage and power capabilities.

More suitable power supplies will be capable of supplying an ablation current at a relatively low voltage, typically below 150V (peak-to-peak), usually being from 50V to 100V. The power will usually be from 5 W to 200 W, usually having a sine wave form, although other wave forms would also be acceptable. Alternatively, the RF energy may be pulsed (e.g., each pulse may have a duration of 0.2 s), which has been shown to provide a more efficient tissue ablation. In general, the amount of power necessary to allow rapid advancement of the biopsy device through tissue will be dictated by the size of the wire 51, 62, 74. Because the size of the wires 51, 62, 74 are relatively small, the amount of power necessary to provide this effect will be relatively low. While power at a level of 20 W will be almost always be sufficient, in most cases power at a level of 10 W, and oftentimes power at a level in the range of about 1 and 10 W, will be sufficient to allow rapid advancement of the cannula, introducer, or cutter.

Power supplies capable of operating within these ranges are available from commercial vendors, such as Boston Scientific Corporation, who markets these power supplies under the trademarks RF2000 (100 W) and RF3000 (200 W). These power supplies have built-in impedance and temperature measurement circuitry that may operate with impedance or temperature sensors located on the distal end of the cannula, introducer, and/or tissue cutter.

Further described herein are methods of sampling tissue. In one embodiment, a sample of tissue is obtained from a patient with an electrosurgical biopsy device. The device can include a cannula having a wire positioned on the distal surface thereof. In one embodiment, the cannula can cooperate with additional sampling components such as, for example, an introducer that can penetrate tissue to move the biopsy device into a target tissue sampling area and/or a tissue cutter that can electrosurgically cut a portion of tissue positioned within a tissue sample chamber.

Prior to inserting the biopsy device into a patient, the introducer can be positioned within an inner lumen of the cannula such that the distal end of the introducer extends distally from the cannula. RF energy can then be delivered to a wire positioned on the distal end of the introducer and the biopsy device can be moved through a tissue mass. The RF energy can ablate tissue and allow the biopsy device to move to a target tissue location.

A number of radiological techniques can be used to confirm the location of the distal end of the biopsy device. For example, an imaging technique, such as x-ray, MRI, CT, PET, SPECT and combinations thereof, can be used to visualize the cannula and/or introducer. In order to facilitate the step of determining the location of the biopsy device, the cannula and/or introducer can include a radio opaque marker or other imageable component.

Once the biopsy device is positioned at the desired location, the user can move the introducer proximally with respect to the cannula to configure the biopsy device in a sampling configuration. RF energy can be delivered to the wire positioned on the distal end of the cannula and the user can advance the cannula such that a portion of tissue enters the inner lumen of the cannula.

In one aspect, an electrosurgical tissue cutter can work with the cannula to cut the portion of tissue in the cannula away from a tissue mass. For example, RF energy can be delivered to a wire positioned on the distal end of the tissue cutter such that the tissue cutter electrosurgically incises the tissue.

As part of the method of sampling tissue, a physician and/or the biopsy device may limit the level of electrosurgical energy provided to each of the cannula, introducer, and/or tissue cutter to a maximum of 20 joules per second to minimize tissue damage, particularly damage to the tissue sample. For example, wires positioned on the cannula, introducer, and/or tissue cutter can receive electrosurgical energy in the range of about 1 to 15 joules per second to prevent tissue damage, or, more preferably, in the range of about 1 to 10 joules per second.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An apparatus for obtaining a sample of tissue from a patient, comprising:
a cannula including an elongated body extending from a proximal end to a distal end and an inner lumen extending from a distal opening for receiving the sample of tissue;
a first wire mated to the distal end of the elongated body and comprising an electrically conductive material; and
an energy source in electrical communication with the first wire and configured to provide low power electrosurgical energy to the first wire.

2. The apparatus of claim 1, wherein the first wire and the cannula do not have any mechanical tissue-cutting surfaces.

3. The apparatus of claim 1, wherein the distal end of the elongate body is defined by a distal surface and the first wire covers less than the full surface area of the distal surface.

4. The apparatus of claim 1, wherein the elongated body has a first wall thickness at the distal opening and the first wire has a cross-sectional dimension transverse to a longitudinal axis of the elongate body which is less than the first wall thickness.

5. The apparatus of claim 1, wherein the diameter of the first wire does not exceed about 75% of the first wall thickness.

6. The apparatus of claim 1, wherein the diameter of the first wire does not exceed about 50% of the first wall thickness.

7. The apparatus of claim 1, wherein the wire is affixed to the distal end of the elongated body using an adhesive.

8. The apparatus of claim 1, wherein the first wire is partially molded into an insulated section of the distal end of the elongated body.

9. The apparatus of claim 1, wherein at least 50% of the surface area of the wire is exposed to allow transmission of energy to the tissue.

10. The apparatus of claim 1, wherein at least 75% of the surface area of the first wire is exposed to allow transmission of energy to the tissue.

11. The apparatus of claim 1, wherein first wire allows the elongate body to penetrate tissue when the electrosurgical energy delivered to the first wire does not exceed about 15 joules per second.

12. The apparatus of claim 1, wherein the first wire is sized to allow tissue penetration when the electrosurgical energy delivered to the first wire is in the range of about 1 to 10 joules per second.

13. The apparatus of claim 1, wherein the first wire is sized to allow tissue penetration when the electrosurgical energy delivered to the first wire is in the range of about 1 to 7 joules per second.

14. The apparatus of claim 1, wherein a distal portion of the elongated body is formed of at least one electrically insulative material.

15. The apparatus of claim 1, wherein an electrically insulative member is positioned between the distal end of the elongated body and the first wire.

16. The apparatus of claim 1, wherein the cannula includes an aperture in a sidewall adapted to receive a tissue cutting device.

17. The apparatus of claim 1, further comprising:
an introducer configured to be inserted into the inner lumen of the cannula and including an elongated body and a blunt distal tip; and
a second wire affixed to the distal tip of the introducer and comprising an electrically conductive material.

18. A system for obtaining a sample of tissue from a patient, comprising:
a cannula, including an elongated body extending from a proximal end to a distal end and an inner lumen extending from a distal opening for receiving the sample of tissue, and a first wire mated to the distal end of the elongated body and comprising an electrically conductive material;
an introducer positioned with inner lumen of the cannula and including an elongated body and a blunt distal tip; and
a tissue cutting device.

19. The system of claim 18, further comprising a second wire affixed to the distal tip of the introducer and comprising an electrically conductive material.

20. The system of claim 19, wherein the second wire has a volume small enough to allow tissue penetration when the electrosurgical energy delivered to the second wire does not exceed about 15 joules per second.

21. The system of claim 19, wherein the second wire is sized to allow tissue penetration when the electrosurgical energy delivered to the second wire is in the range of about 1 to 10 joules per second.

22. The system of claim 19, wherein the second wire is sized to allow tissue penetration when the electrosurgical energy delivered to the second wire is in the range of about 1 to 7 joules per second.

23. The system of claim 18, further comprising a tissue cutter configured to enter into the inner lumen through an aperture proximate to the distal end of the elongated body of the cannula and a third wire positioned on the tissue cutter for delivering low power electrosurgical energy to tissue.

24. The system of claim 23, wherein the tissue cutter comprises a resilient material.

25. The system of claim 23, wherein the third wire has a volume small enough to allow tissue penetration when the electrosurgical energy delivered to the third wire does not exceed 15 joules per second.

26. A method for obtaining a sample of tissue from a patient, comprising:
providing a cannula including an elongated body extending from a proximal end to a distal end, an inner lumen extending from a distal opening for receiving the sample of tissue, and a first wire mated to the distal end of the elongated body and comprising an electrically conductive material;
providing electrosurgical energy of less than about 10 joules per second to the first wire; and
collecting a tissue sample in the inner lumen.

27. The method of claim 26, further comprising the steps of:
providing an introducer including an elongated body, a blunt distal tip, and a second wire mated to the distal tip of the introducer;
providing electrosurgical energy to the second wire; and
moving the cannula and introducer through a tissue mass.
